(19) United States
(12) Patent Application Publication
(10) Pub. No.: US 2026/0241175 A1
(43) Pub. Date: Aug. 20, 2026

(54) SYSTEMS AND METHODS FOR TREATING MOTOR DYSFUNCTION, INCLUDING BY SELECTIVELY ACTIVATING AND/OR SUPPRESSING MOTOR NEURONS AND/OR MOTOR RESPONSES

(71) Applicant: Nevro Corp., Redwood City, CA (US)

(72) Inventors: Dongchul Lee, Agua Dulce, CA (US); Kwan Yeop Lee, Mississauga (CA)

(21) Appl. No.: 19/649,270

(22) Filed: Apr. 16, 2026

Related U.S. Application Data
(62) Division of application No. 18/664,524, filed on May 15, 2024.
Application No. 18/664,524 is a continuation of application No. PCT/US2022/049958, filed on Nov. 15, 2022.
(60) Provisional application No. 63/279,608, filed on Nov. 15, 2021.

(51) Int. Cl. A61N 1/36; A61N 1/05; A61N 1/372

(52) U.S. Cl. CPC A61N 1/36171; A61N 1/0551; A61N 1/36062; A61N 1/37217

(57) ABSTRACT

The present technology is directed generally to electrical stimulation and associated systems and methods for preferentially and/or selectively activating and suppressing motor neurons and/or motor responses, such as to treat a motor dysfunction in patients. For example, in some embodiments high frequency electrical stimulation can be administered to a target neural population via an implanted signal delivery device to induce a motor response in a first muscle, and low frequency electrical stimulation can be administered to the target neural population via the same signal delivery device to induce a motor response in a second muscle that is different than the first muscle.

### CROSS-REFERENCE TO RELATED APPLICATIONS

The present application is a divisional of U.S. Patent Application No. 18/664,524, filed May 15, 2024 and published as U.S. 2024-0390681, which is a continuation of International Patent Application No. PCT/US22/49958, filed November 15, 2022, which claims priority to U.S. Provisional Application No. 63/279,608, filed November 15, 2021, the disclosure of each of which is incorporated by reference herein in its entirety.

### FIELD

The present technology is directed towards electrically modulating motor neural pathways to treat motor dysfunction.

### BACKGROUND

Neurological stimulators have been developed to treat pain, movement disorders, functional disorders, spasticity, cancer, cardiac disorders, and various other medical conditions. Implantable neurological stimulation systems generally have an implantable signal generator and one or more leads that deliver electrical pulses to neurological tissue or muscle tissue. For example, several neurological stimulation systems for spinal cord stimulation (SCS) have cylindrical leads that include a lead body with a circular cross-sectional shape and one or more conductive rings (e.g., contacts) spaced apart from each other at the distal end of the lead body. The conductive rings operate as individual electrodes and, in many cases, the SCS leads are implanted percutaneously through a needle inserted into the epidural space, with or without the assistance of a stylet. In other systems, the electrodes are carried by a paddle that is implanted via a laminotomy.

Once implanted, the signal generator applies electrical pulses to the electrodes, which in turn modify the function of the patient's nervous system, such as by altering the patient's responsiveness to sensory stimuli and/or altering the patient's motor-circuit output. In SCS therapy for the treatment of pain, for example, the signal generator applies electrical pulses to the spinal cord via the electrodes.

### BRIEF DESCRIPTION OF THE DRAWINGS

FIG. 1 is a partially schematic illustration of an implantable spinal cord modulation system positioned at a patient's spine to deliver therapeutic signals in accordance with some embodiments of the present technology.

FIG. 2A is a partially schematic, cross-sectional illustration of a patient's spine, illustrating representative locations for implanted lead bodies in accordance with some embodiments of the present technology.

FIG. 2B is a partially schematic, cross-sectional illustration of a patient's spine, illustrating additional representative locations for implanted lead bodies in accordance with some embodiments of the present technology.

FIG. 3 is a schematic illustration of a representative lead body suitable for providing modulation to a patient in accordance with several embodiments of the present technology.

FIG. 4A is a line graph illustrating a motor response of a rat to a high frequency electrical signal applied to the rat's spinal cord. FIG. 4B is a line graph illustrating a motor response of the rat to a low frequency electrical signal applied to the rat's spinal cord.

FIG. 5A is a line graph illustrating the thigh motor response of a rat to a first high frequency signal and to a second high frequency signal administered after the first high frequency signal. FIG. 5B is a line graph illustrating the toe motor response of the rat to a first low frequency signal and to a second low frequency signal administered after the first low frequency signal.

FIG. 6A is a line graph illustrating a thigh motor response of a rat to a high frequency signal and a toe motor response of the rat to a low frequency signal administered after the high frequency signal. FIG. 6B is a line graph illustrating a toe motor response of the rat to a low frequency signal and a thigh motor response to a high frequency signal administered after the low frequency signal.

FIG. 7 is a line graph comparing the inhibitory effect of a high frequency signal and a low frequency signal on a toe motor neuron of a rat.

FIG. 8 is a block diagram illustrating a method for treating a patient in accordance with embodiments of the present technology.

FIG. 9 is a block diagram illustrating another method for treating a patient in accordance with embodiments of the present technology.

FIG. 10 is a block diagram illustrating yet another method for treating a patient in accordance with embodiments of the present technology.

### DETAILED DESCRIPTION

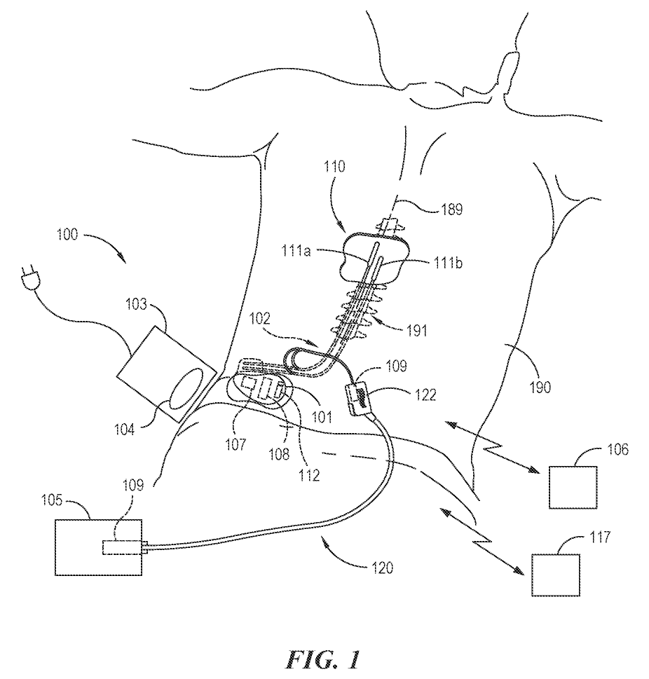

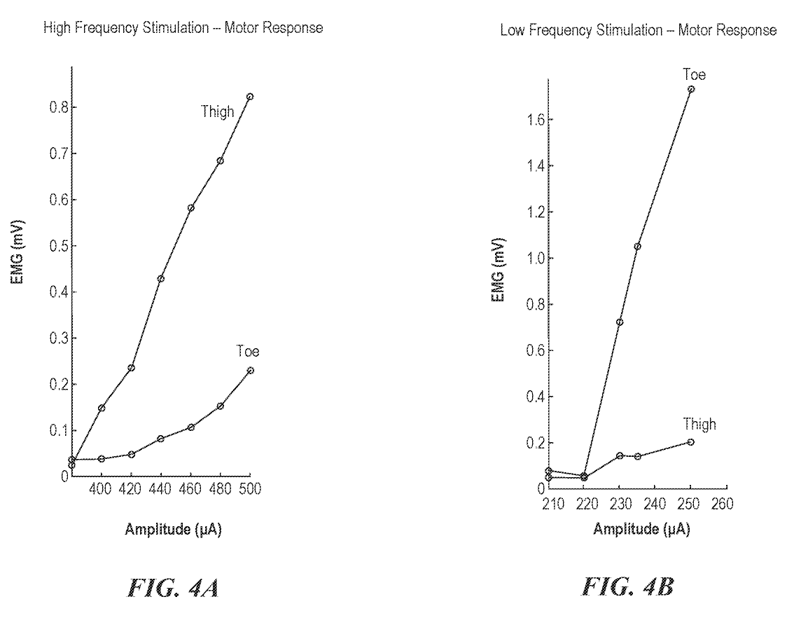

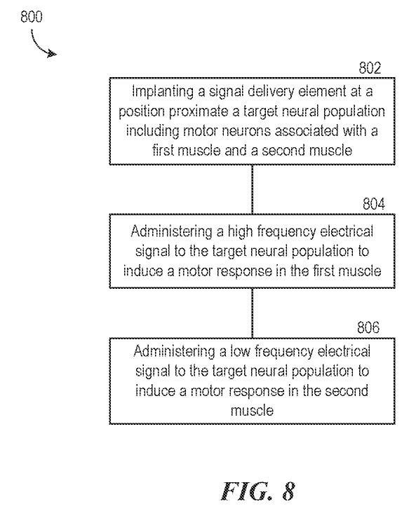

This Detailed Description includes the following headers and sections, which are provided for convenience only and do not the scope or meaning of the claimed present technology: Definitions of selected terms are provided under Heading 1.0 (“Definitions”); General aspects of the present technology are described below under Heading 2.0 (“Overview of Present Technology”); Representative treatment systems and their characteristics are described under Heading 3.0 (“System Characteristics”) with reference to FIGS. 1-3; Use of electrical stimulation to selectively activate and inhibit motor neurons, including animal data, are described under Heading 4.0 (“Preferential and/or Selective Activation and Inhibition of Motor Neurons”) with reference to FIGS. 4A-7; Representative methods of selectively activating or inhibiting motor neurons in accordance with the present technology are described under Heading 5.0 (“Methods for Preferentially and/or Selectively Activating or Inhibiting Motor Neurons”) with reference to FIG. 8-10; Representative clinical applications of the present technology are described under Heading 6.0 (“Representative Clinical Applications”); Representative electrical signal delivery parameters are described under Heading 7.0 (“Representative Signal Delivery Parameters”); and Representative examples are described under Heading 8.0 (“Representative Examples”).

### 1.0 Definitions

Unless otherwise stated, the terms “generally,” “about,” and “approximately” refer to values within 10% of a stated value. For example, the use of the term “about 100” refers to a range of 90 to 110, inclusive. In instances where relative terminology is used in reference to something that does not include a numerical value, the terms are given their ordinary meaning to one skilled in the art.

As used herein, and unless otherwise noted, the terms “modulate,” “modulation,” “stimulate,” and “stimulation” refer generally to signals that have an inhibitory, excitatory, and/or other effect on a target neural population. Accordingly, a spinal cord “stimulator” can have an inhibitory effect on certain neural populations. Moreover, the use of the terms “suppress” and “inhibit” in relation to a therapy signal's effect on a neuron refers to a reduction in the neuron's firing rate relative to the neuron's baseline firing rate in the absence of the therapy signal, and does not necessarily refer to a complete elimination of action potentials in the neuron.

As used herein, the terms “neuromodulation signal”, “electrical therapy signal,” “electrical signal,” “therapy signal,” “signal,” and other associated terms are used interchangeably and generally refer to an electrical signal that can be characterized by one more parameters, such as frequency, pulse width, and/or amplitude.

As used herein, the term “preferentially” when used in the context of “preferentially” activating a first subset of neurons and/or a first muscle relative to a second subset of neurons and/or a second muscle refers to activating the first subset of neurons and/or the first muscle to a greater degree than the second subset of neurons and/or the second muscle. The second subset of neurons and/or the second muscle are either not activated or are activated to a lesser degree than the first subset of neurons and/or the first muscle. As used herein, the term “selectively” when used in the context of “selectively” activating a first subset of neurons and/or a first muscle relative to a second subset of neurons and/or a second muscle refers to activating the first subset of neurons and/or the first muscle without activating (e.g., to a patient-detectable degree) the second subset of neurons and/or the second muscle.

As used herein, the term “high frequency” when used to describe an electrical signal refers to an electrical signal having a frequency between about 1.2 kHz and about 500 kHz, unless specifically stated otherwise. As used herein, the term “low frequency” when used to describe an electrical signal refers to an electrical signal having a frequency of less than about 1.2 kHz, unless specifically stated otherwise.

As used herein, the term “pulse width” refers to the width of any phase of a repeating pulse, such as the portion of a pulse at a given polarity, unless explicitly described otherwise. For example, the use of the term pulse width with respect to a signal having bi-phasic pulses can refer to the duration of an anodic pulse phase or a cathodic pulse phase. The use of the term pulse width with respect to a signal having monophasic pulses can refer to the duration of the monophasic pulse phase.

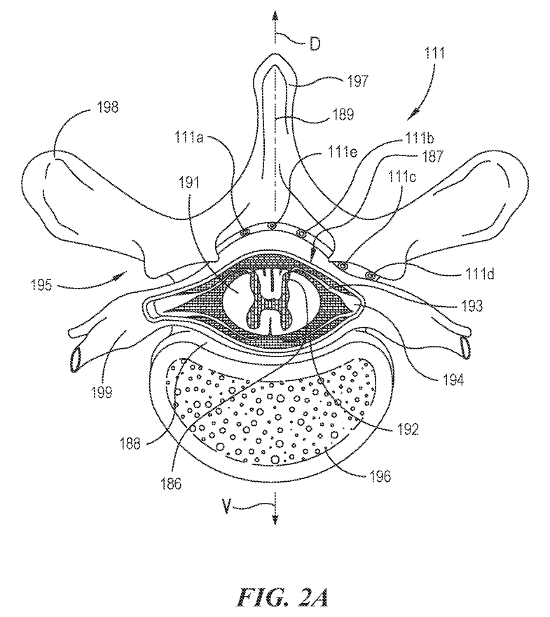

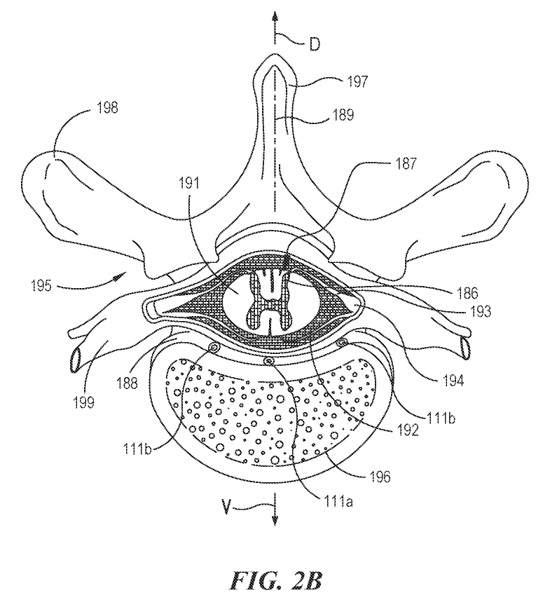

As used herein, “proximate a spinal cord region” refers to the placement of a signal delivery element such that it can deliver electrical stimulation to a neural population associated with the spinal cord or associated nervous system structures. For example, “proximate a spinal cord region” includes, but is not limited to, the relative lead positions described and shown in FIGS. 2A and 2B, as well as other positions not expressly described herein.

As used herein, “proximate a target neural population” refers to the placement of a signal delivery element such that it can deliver electrical stimulation to the target neural population. For example, if the target population includes neurons in the spinal cord at a given vertebral level, “proximate the target neural population” includes, but is not limited to, the relative lead positions described and shown in FIGS. 2A and 2B at the given vertebral level, as well as other positions not expressly described herein. As another example, if the target population includes neurons in the patient's cortex (e.g., motor cortex), “proximate the target neural population” includes, but is not limited to, leads positioned in or on the patient's cortex.

### 2.0 Overview of the Present Technology

The present technology is directed generally to electrical stimulation and associated systems and methods for preferentially and/or selectively activating and suppressing motor neurons and/or motor responses, such as to treat a motor dysfunction in patients. For example, in some embodiments high frequency electrical stimulation can be administered to a target neural population including a first subset of motor neurons associated with a first muscle and a second subset of motor neurons associated with a second muscle. The high frequency electrical stimulation can preferentially and/or selectively activate or excite the first subset of motor neurons to induce a motor response in the first muscle. In some embodiments, the high frequency signal activates the first subset of motor neurons without activating the second subset of motor neurons, and therefore without inducing a motor response (or at least without inducing a patient-discernable motor response) in the second muscle. Indeed, in some embodiments the high frequency electrical signal may even suppress or inhibit the second subset of motor neurons. In some embodiments, a low frequency electrical signal can be administered to the target neural population of neurons via the same signal delivery device that administered the high frequency electrical signal. The low frequency electrical signal can activate or excite the second subset of motor neurons to induce a motor response in the second muscle. In some embodiments, the low frequency signal activates the second subset of motor neurons without activating the first subset of motor neurons, and therefore without inducing a motor response (or at least without inducing a patient-discernable motor response) in the first muscle. Accordingly, the present technology can be used to preferentially and/or selectively induce a motor response in different muscles simply by manipulating the frequency of the electrical signal, reducing or even eliminating the need to reposition the signal delivery device.

Specific details of certain embodiments of the disclosure are described below with reference to methods for modulating one or more target neural populations (e.g., nerves) or sites of a patient, and associated implantable structures for providing the modulation. Although selected embodiments are described below with reference to modulating the dorsal column, dorsal horn, dorsal root, dorsal root entry zone, ventral column, ventral horn, and/or other particular regions of the spinal column, the modulation may in some instances be directed to other neurological structures and/or target neural populations of the spinal cord and/or other neurological tissues. For example, some embodiments may include modulating brain tissue, including the cortex (e.g., motor cortex) and/or deep brain structures. Some embodiments can have configurations, components or procedures different than those described in this section, and other embodiments may eliminate particular components or procedures. A person of ordinary skill in the relevant art, therefore, will understand that the present disclosure may include other embodiments with additional elements, and/or may include other embodiments without several of the features shown and described below with reference to FIGS. 1-10.

### 3.0 System Characteristics

FIG. 1 schematically illustrates a representative patient therapy system 100 for treating a patient's motor, sensory, and/or other functioning, arranged relative to the general anatomy of the patient's spinal column 191. The system 100 can include a signal generator 101 (e.g., an implanted or implantable pulse generator or IPG), which can be implanted subcutaneously within a patient 190 and coupled to one or more signal delivery elements or devices 110. The signal delivery elements or devices 110 can be implanted within the patient 190, at or off the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms signal delivery device, signal delivery element, lead, and/or lead body include any of a number of suitable substrates and/or supporting members that carry electrodes/devices for providing therapy signals to the patient 190. For example, the lead or leads 111 can include one or more electrodes or electrical contacts that direct electrical signals into the patient's tissue, e.g., to provide for therapeutic relief. In some embodiments, the signal delivery elements 110 can include structures other than a lead body (e.g., a paddle) that also direct electrical signals and/or other types of signals to the patient 190, e.g., as disclosed in U.S. Patent Application Publication No. 2018/0256892, incorporated herein by reference in its entirety. For example, paddles can be more suitable for patients with spinal cord injuries that result in scarring or other tissue damage that impedes cylindrical leads.

In some embodiments, one signal delivery device can be implanted on one side of the spinal cord midline 189, and a second signal delivery device can be implanted on the other side of the spinal cord midline 189. For example, the first and second leads 111a, 111b shown in FIG. 1 can be positioned just off the spinal cord midline 189 (e.g., about 1 mm offset) in opposing lateral directions so that the two leads 111a, 111b are spaced apart from each other by about 2 mm. In some embodiments, the leads 111 can be implanted at a vertebral level ranging from, for example, about T1 to about T12, or from about T4 to about T12. In some embodiments, one or more signal delivery devices can be implanted at other vertebral levels, e.g., as disclosed in U.S. Patent No. 9,327,121, incorporated herein by reference in its entirety.

The signal generator 101 can transmit signals (e.g., electrical signals) to the signal delivery elements 110 that excite and/or suppress target nerves. The signal generator 101 can include a machine-readable (e.g., computer-readable or controller-readable) medium containing instructions for generating and transmitting suitable therapy signals, such to perform the methods described below with respect to FIGS. 8-10. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on, or in computer-readable media located at the pulse generator 101 and/or other system components. Further, the pulse generator 101 and/or other system components can include dedicated hardware, firmware, and/or software for executing computer-executable instructions that, when executed, perform any one or more methods, processes, and/or sub-processes described herein and/or in the materials incorporated herein by reference. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1, or in multiple housings. For example, the signal generator can include some components that are implanted (e.g., a circuit that directs signals to the signal delivery device 110), and some that are not (e.g., a power source). The computer-executable instructions can be contained on one or more media that are implanted within the patient and/or positioned external to the patient, depending on the embodiment.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1 for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Patent No. 8,355,797, incorporated herein by reference in its entirety.

In some embodiments, the signal generator 101 and/or signal delivery devices 110 can obtain power to generate the therapy signals from an external power source 103. For example, the external power source 103 can by-pass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use.

In some embodiments, the signal generator 101 can obtain the power to generate therapy signals from an internal power source, in addition to or in lieu of the external power source 103. For example, the implanted signal generator 101 can include a non-rechargeable battery or a rechargeable battery to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110, e.g., during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator 105 and can be used by the clinician to help determine what parameters to vary. In a typical process, the practitioner uses a cable assembly 120 to temporarily connect the trial modulator 105 to the signal delivery device 110. The practitioner can test the efficacy of the signal delivery devices 110 in an initial position. The practitioner can then disconnect the cable assembly 120 (e.g., at a connector 122), reposition the signal delivery devices 110, and reapply the electrical signals. This process can be performed iteratively until the practitioner obtains the desired position for the signal delivery devices 110. Optionally, the practitioner can move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, incorporated by reference herein in its entirety.

After the signal delivery elements 110 are implanted, the patient 190 can receive therapy via signals generated by the trial modulator 105, generally for a limited period of time. During this time, the patient wears the cable assembly 120 and the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. In still further embodiments, the signal generator 101 can be implanted without first undergoing a trial period. Once the implantable signal generator 101 has been positioned within the patient 190, the therapy programs provided by the signal generator 101 can still be updated remotely via a wireless physician's programmer 117 (e.g., a physician's laptop, a physician's remote or remote device, etc.) and/or a wireless patient programmer 106 (e.g., a patient's laptop, patient's remote or remote device, etc.). Generally, the patient 190 has control over fewer parameters than does the practitioner. For example, the capability of the patient programmer 106 can be limited to starting and/or stopping the signal generator 101, and/or adjusting the signal amplitude within a present amplitude range. The patient programmer 106 can be configured to accept inputs corresponding to pain relief, motor functioning and/or other variables, such as medication use. Accordingly, more generally, embodiments of the present technology include receiving patient feedback, via a sensor, that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude, or delivery location.

FIG. 2A is a cross-sectional illustration of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIG. 2A implanted in a single patient. In addition, for purposes of illustration, the leads 111 are shown as elongated leads however, leads 111 can be paddle leads. In actual use, any given patient will likely receive fewer than all the leads 111 shown in FIG. 2A.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193, and dorsal root ganglia 194. The dorsal roots 193 enter the spinal cord 191 at the dorsal root entry region 187, and communicate with dorsal horn neurons located at the dorsal horn 186. In some embodiments, the first and second leads 111a, 111b are positioned just off the spinal cord midline 189 (e.g., about 1 mm offset) in opposing lateral directions so that the two leads 111a, 111b are spaced apart from each other by about 2 mm, as discussed above. In some embodiments, a lead or pairs of leads can be positioned at other locations, e.g., toward the outer edge of the dorsal root entry region 187 as shown by a third lead 111c, or at the dorsal root ganglia 194, as shown by a fourth lead 111d, or approximately at the spinal cord midline 189, as shown by a fifth lead 111e.

In some embodiments, the leads can be positioned on the ventral side of the spinal cord to better target or access certain motor neuron populations. FIG. 2B, for example, illustrates a signal delivery device 111f positioned at a ventral location within the vertebral foramen 188 (e.g., within the patient's spinal canal), at or approximately at the spinal cord midline 189. Similarly, in another embodiment, one or more leads 111g are positioned off the spinal cord midline 189, laterally or bilaterally. From these locations, the lead(s) 111 can direct therapeutic signals to ventral neural populations at the spinal cord 191 itself, or to neural populations in the region of the spinal cord, but off the spinal cord itself, e.g., the laterally-positioned ventral roots 192.

In some embodiments, the devices and systems of the present technology include features other than those described herein. For example, one lead 111 to six leads 111 can be positioned generally end-to-end at or near the patient's midline M and span vertebral levels from about T4 to about T12. In some embodiments, two, three, or four leads 111 are positioned end-to-end at or near the patient's midline from T4 to T12. In some embodiments, the leads 111 and/or other signal delivery devices can have locations other than those expressly shown herein. For example, one or more signal delivery devices can be positioned at the dorsal side of the spinal cord 191. In addition, the devices and systems of the present technology can include more than one internal stimulator and/or more than one external stimulator that can be configured for wireless stimulation, such as by using electromagnetic waves.

Several aspects of the technology are embodied in computing devices, e.g., programmed/programmable pulse generators, controllers and/or other devices. The computing devices on/in which the described technology can be implemented can include one or more central processing units, memory, input devices (e.g., input ports), output devices (e.g., display devices), storage devices, and network devices (e.g., network interfaces). The memory and storage devices are computer-readable media that can store instructions that implement the technology. In some embodiments, the computer readable media are tangible media. In some embodiments, the data structures and message structures can be stored or transmitted via an intangible data transmission medium, such as a signal on a communications link. Various suitable communications links can be used, including but not limited to a local area network and/or a wide-area network.

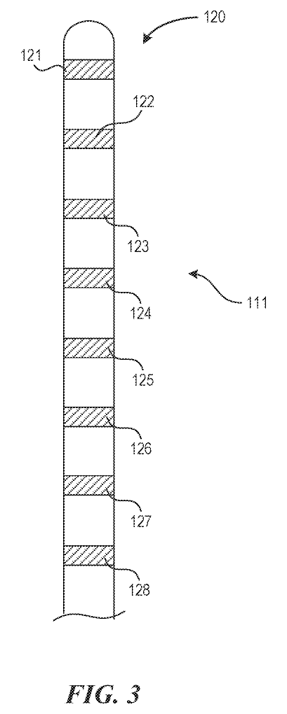

FIG. 3 is a partially schematic illustration of a representative lead body 211 that can be used to apply modulation to a patient in accordance with any of the foregoing embodiments. In general, the lead body 211 includes a multitude of electrodes or contacts 220. When the lead body 211 has a circular cross-sectional shape, as shown in FIG. 3, the contacts 220 can have a generally ring-type shape and can be spaced apart axially along the length of the lead body 211. In a particular embodiment, the lead body 211 can include eight contacts 220, identified individually as first, second, third . . . eighth contacts 212, 222, 223 . . . 228. In general, one or more of the contacts 220 are used to provide signals, and another one or more of the contacts 220 provide a signal return path. Accordingly, the lead body 211 can be used to deliver monopolar modulation (e.g., if the return contact is spaced apart significantly from the delivery contact), or bipolar modulation (e.g., if the return contact is positioned close to the delivery contact and in particular, at the same target neural population as the delivery contact). In still further embodiments, the pulse generator 101 (FIG. 1) can operate as a return contact for monopolar modulation.

### 4.0 Preferential and/or Selective Activation and Inhibition of Motor Neurons

As described in detail below, the present technology is generally directed to systems for and methods of administering electrical stimulation to (1) preferentially and/or selectively excite or inhibit motor neurons, and (2) preferentially and/or selectively induce or prevent motor responses in muscles associated with the motor neurons. For example, as described below, the present technology includes frequency-dependent activation of certain subsets of motor neurons to preferentially induce a motor response in a first muscle while generally avoiding inducing a motor response in a second muscle.

FIG. 4A is a line graph illustrating a motor response of a rat to a high frequency electrical stimulation signal applied to the rat's spinal cord, and FIG. 4B is a line graph illustrating the motor response of the rat to a low frequency electrical signal applied to the rat's spinal cord at the same location (e.g., using the same electrodes) as the high frequency electrical stimulation signal. In particular, the x-axis of the line graphs reflects the amplitude of the electrical signals, and the y-axis of the line graphs of FIGS. 4A and 4B quantifies an electromyogram (EMG) response to the high and low frequency signals, respectively. As shown, EMG responses were obtained at both thigh muscle and toe muscle in response to both the high frequency signal (FIG. 4A) and the low frequency signal (FIG. 4B).

Referring first to FIG. 4A, the high frequency electrical signal was a bi-phasic signal having a frequency of 10 kHz. The signal had a 20 microsecond anodic pulse phase, a 20 microsecond interphase interval, a 20 microsecond cathodic pulse phase, and a 40 microsecond interpulse interval. The signal was administered for 3 milliseconds, followed by a 5 second off-period. As shown in FIG. 4A, the high frequency signal preferentially activated the thigh muscle (and motor neurons associated with the thigh muscle) compared to the toe muscle (and motor neurons associated with the toe muscle). However, for both the thigh muscle and the toe muscle, the induced response increased generally linearly as the amplitude of the electrical signal increased.

Referring next to FIG. 4B, the low frequency electrical signal was a bi-phasic signal having one pulse every five seconds (e.g., a frequency of 0.2 Hz). The pulses had a 100 microsecond anodic pulse phase, a 20 microsecond interphase interval, and a 100 microsecond cathodic pulse phase. As shown in FIG. 4B, the low frequency signal preferentially activated the toe muscle (and motor neurons associated with the toe muscle) compared to the thigh muscle (and motor neurons associated with thigh muscle). Of note, this is the opposite of the activation pattern induced with the high frequency electrical signal, shown in FIG. 4A.

Taken together, FIGS. 4A and 4B demonstrate that different motor neurons and/or different muscle groups can be preferentially excited/activated simply by varying the frequency or other parameters of an electrical signal. Of note, this can be done without changing the electrode (or pair of electrodes) that is used (e.g., activated) to deliver the signal, and without repositioning (e.g., intra-operatively repositioning) the signal delivery device (e.g., the lead, electrode, etc.). As set forth below, this is expected to simplify the process of preferentially activating various motor neurons and/or muscles relative to conventional techniques. In some embodiments, it is also expected to enable use of smaller signal delivery devices (e.g., due to requiring fewer electrodes), which may result in less invasive procedures. Additional details regarding the clinical application of the present technology, including potential benefits, are described below in Sections 5.0 and 6.0.

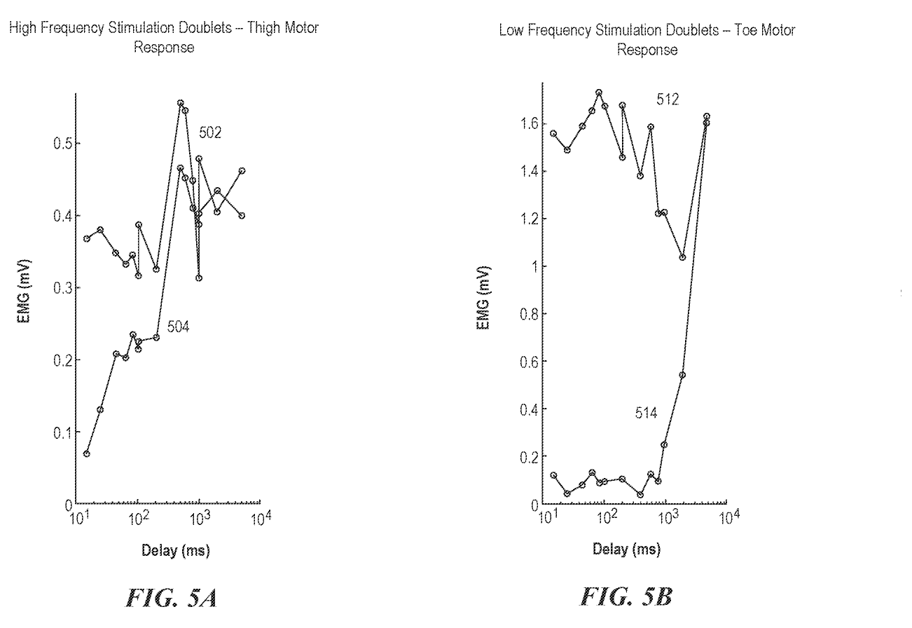

The motor response induced by the high and low frequency signals shown in FIGS. 4A and 4B may be subject to a recovery time following stimulation. FIG. 5A is a line graph illustrating a thigh motor response of a rat to a first high frequency signal and to a second high frequency signal administered after the first high frequency signal (also referred to as a “high frequency doublet”). FIG. 5B is a line graph illustrating a toe motor response of a rat to a first low frequency signal and to a second low frequency signal administered after the first low frequency signal (also referred to as a “low frequency doublet”). The data in FIGS. 5A and 5B were obtained by administering the first (e.g., leading) signal, ceasing to deliver the first (e.g., leading) signal for a delay period, and administering the second (e.g., following) signal. For both FIGS. 5A and 5B, the x-axis reflects the time period of the delay between the first and second signals, and the y-axis reflects the EMG response to the signals. The thigh motor response to the first high frequency signal is shown in FIG. 5A by line 502 and the thigh motor response to the second high frequency signal is shown in FIG. 5A by line 504. The toe motor response to the first low frequency signal is shown in FIG. 5B by line 512 and the toe motor response to the second low frequency signal is shown in FIG. 5B by line 514.

Referring first to FIG. 5A, the first high frequency signal was a 10 kHz signal applied for approximately 3 milliseconds. The second high frequency signal was also a 10 kHz signal applied for approximately 3 milliseconds. The first and second high frequency signals were separated by a variable delay of between 10 milliseconds and 10 seconds. As shown by the line 502 in FIG. 5A, the first high frequency signal induced a generally consistent motor response in the rat's thigh muscle (and associated motor neurons), varying between about 0.3-0.6 mV. This is consistent with the data illustrated in FIG. 4A. However, the response induced by the second high frequency signal (shown by the line 504) was suppressed as a function of the time delay between the application of the first signal and the second signal. For example, when the first and second signals were administered with a relatively short delay separating the two (e.g., less than about 500 milliseconds), the motor response to the second signal was generally suppressed or lower than the motor response to the first signal (e.g., less than about 0.3 mV). However, when the first and second signals were administered with a relatively long delay separating the two (e.g., greater than about 1 second), the motor response to the second signal was generally the same as (and thus not suppressed by) the motor response to the first signal (e.g., between about 0.3-0.6 mV).

A similar result was observed following administration of the low frequency doublet. The first low frequency signal included a single pulse having a 100 microsecond anodic pulse phase, a 20 microsecond interphase interval, and a 100 microsecond cathodic pulse phase. The second low frequency signal was generally the same as the first low frequency signal. The first and second low frequency signals were separated by a variable delay of between 10 milliseconds and 10 seconds. As shown by the line 512 in FIG. 5B, the first low frequency signal induced a generally consistent motor response in the rat's toe muscle (and associated motor neurons), varying between about 1.0-1.6 mV. This is consistent with the data illustrated in FIG. 4B. However, the response induced by the second low frequency signal (shown by the line 514) was suppressed as a function of the time delay between the application of the first signal and the second signal. For example, when the first and second signals were administered with a relatively short delay separating the two (e.g., less than about 500 milliseconds), the motor response to the second signal was generally suppressed or lower than the motor response to the first signal (e.g., less than about 0.6 mV). However, when the first and second signals were administered with a relatively long delay separating the two (e.g., greater than about 1 second), the motor response to the second signal was generally the same as (and thus not suppressed by) the motor response to the first signal (e.g., greater than about 1 mV).

Without being bound by theory, the time-delay proportional inhibition of a motor response to a second stimulus shown in both FIGS. 5A and 5B is consistent with similar time-delay proportional inhibition observed when testing a human H-reflex or posterior root-muscle (PRM) reflex with paired pulses. Accordingly, one potential mechanism explaining the inhibition seen in FIGS. 5A and 5B is that the first signal or pulse induces presysnaptic inhibition and homosynaptic depression, with a time-delayed recovery. Additional details regarding the time-delay recovery of the human H-reflex and PRM-reflex are described in the publication by Hofstoetter et al., entitled “Recovery Cycles of Posterior Root-Muscle Reflexes Evoked by Transcutaneous Spinal Cord Stimulation and of the H reflex in Individuals with Intact and Injured Spinal Cord,” and published in PLoS One, 14(12): e0227057, the disclosure of which is incorporated by reference herein in its entirety.

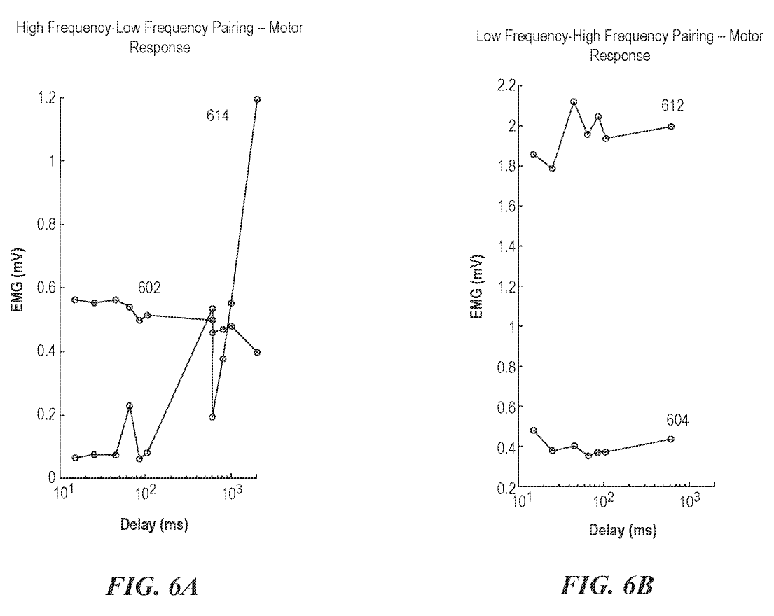

Mixed frequency doublets (e.g., high frequency followed by low frequency) were also tested to determine the effect, if any, on motor neuron function. FIG. 6A is a line graph illustrating a thigh motor response of a rat to a high frequency signal and a toe motor response of the rat to a low frequency signal administered after the high frequency signal. FIG. 6B is a line graph illustrating the toe motor response of the rat to a low frequency signal and the thigh motor response of the rat to a high frequency signal administered after the low frequency signal. For both FIGS. 6A and 6B, the x-axis reflects the time period of the delay between the first (leading) signal and the second (following) signal, and the y-axis reflects the EMG response to the signals. The thigh motor response to the high frequency signal is shown in FIG. 6A by line 602, and the toe motor response to the low frequency signal administered after the high frequency signal is shown in FIG. 6A by line 614. The toe motor response to the low frequency signal is shown in FIG. 6B by line 612, and the thigh motor response to the high frequency signal administered after the low frequency signal is shown in FIG. 6B by line 604. The high frequency signal was generally the same as those described with respect to FIGS. 4A and 5A, and the low frequency signal was generally the same as those described with respect to FIGS. 4B and 5B.

Referring first to FIG. 6A, and as shown by the line 602, the high frequency signal induced a generally consistent motor response in the rat's thigh muscle (and associated motor neurons). This is consistent with the data illustrated in FIGS. 4A and 5A. However, as shown by the line 614, the response in toe muscle to the low frequency signal was suppressed as a function of the time delay between the application of the high frequency signal and the low frequency signal. For example, when the high frequency signal and the low frequency signal were administered with a relatively short delay separating the two (e.g., less than about 100 milliseconds), the toe motor response to the low frequency signal was generally suppressed, even though the high frequency signal did not induce the toe motor response (or at least did not induce a substantial toe motor response). However, when the high frequency signal and low frequency signal were administered with a relatively long delay separating the two (e.g., greater than about 1 second), the toe motor response to the low frequency signal was not suppressed (or at least not substantially suppressed) by the high frequency signal. The data in FIG. 6A therefore demonstrate that a high frequency signal that activates thigh muscle can inhibit/suppress toe muscle without first activating the toe muscle.

Referring next to FIG. 6B, and as shown by the line 612, the low frequency signal induced a generally consistent motor response in the rat's toe muscle (and associated motor neurons). This is consistent with the data illustrated in FIGS. 4B and 5B. However, unlike when the high frequency signal was administered first (e.g., as described with respect to FIG. 6A), the low frequency signal did not inhibit or suppress motor response to a subsequently delivered high frequency signal. For example, as shown by line 604 in FIG. 6B, the thigh motor response to the high frequency signal delivered after the low frequency signal was generally consistent, and was not affected by the time delay between the low frequency signal and the high frequency signal. Moreover, the magnitude of the motor response (as measured by the EMG) was generally similar to the magnitude seen when the high frequency signal was administered before the low frequency signal (note the different scale of the y-axes for FIGS. 6A and 6B). Accordingly, the data in FIG. 6B demonstrates that a low frequency signal that activates the toe muscle does not inhibit/suppress the thigh muscle activation. This is in contrast with the high frequency signal inhibiting activation of the toe muscle, described above with respect to FIG. 6A. Additional details regarding the clinical application and potential benefits of the effects demonstrated in FIGS. 6A and 6B are described below in Sections 5.0 and 6.0.

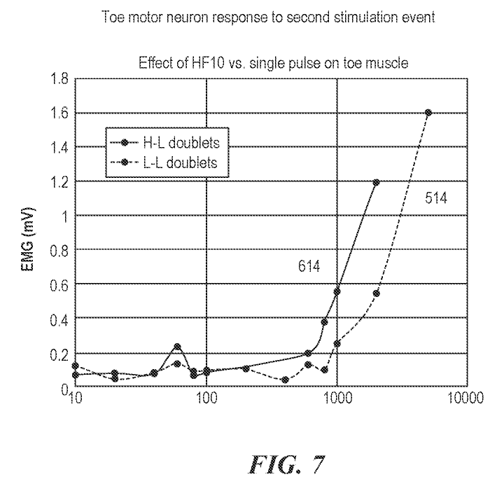

FIG. 7 further demonstrates the inhibitory effect high frequency signals have on motor responses that are generally activated by low frequency signals (e.g., the toe motor response). In particular, line 514 (which is the same as line 514 of FIG. 5B) illustrates the toe muscle response to a second low frequency signal of a low frequency doublet, described with respect to FIG. 5B. Line 614 (which is the same as line 614 of FIG. 6A) illustrates the toe muscle response to a low frequency signal administered after a high frequency signal, described with respect to FIG. 5A. The suppressive effects shown by lines 514 and 614 are generally the same. This means that the suppressive effects on the toe muscle can be achieved by either high frequency stimulation or low frequency stimulation. However, the suppression using high frequency stimulation can be attained without first activating the motor neurons.

Without being bound by theory, one potential mechanism of action explaining the high frequency signal's suppressive effect on the toe muscle is that the high frequency signal activated certain inhibitory circuits associated with the toe muscle, but did not activate excitatory circuits associated with the toe muscle. For example, high frequency signals may activate “recurrent inhibition” and “presynaptic inhibition” circuits to drive inhibition of the toe muscle motor neurons, but without activating motor neurons. As described in detail below in Sections 5.0 and 6.0, this may enable a single high frequency signal to activate a first muscle (e.g., the thigh muscle) while simultaneously suppressing a second muscle (e.g., the toe muscle), without needing to first activate the second muscle.

### 5.0 Methods for Preferentially and/or Selectively Activating or Inhibiting Motor Neurons

The present technology further includes methods for preferentially and/or selectively activating and/or inhibiting motor neurons and/or motor responses to, e.g., treat motor dysfunction in a patient. For example, FIG. 8 is a block diagram illustrating a method 800 for treating a patient in accordance with embodiments of the present technology. Some or all of the operations in the method 800 can be performed by a processor executing instructions stored on one or more elements of a patient treatment system.

The method 800 can begin at block 802 by implanting a signal delivery element at a position proximate a target neural population, the target neural population including motor neurons associated with a first muscle and a second muscle. The first and second muscles can be part of different muscle groups (e.g., a thigh muscle and a toe muscle). The target neural population may include neurons positioned in the patient's brain and/or spinal cord. The motor neurons associated with the first and second muscles do not necessarily directly innervate the first and second muscles (although in some embodiments they may), but rather can include neurons “upstream” of the motor neurons that directly innervate the first and second muscles. The signal delivery element can include any of the signal delivery elements described herein and/or other suitable signal delivery elements known in the art, and may include one or more electrodes for delivering electrical signals to the target neural population.

The method 800 can continue at block 804 by administering a high frequency electrical signal to the target neural population to induce (e.g., selectively induce, preferentially induce, etc.) a motor response in the first muscle. The high frequency signal can have a frequency in a frequency range of from about 1.2 kHz to about 500 kHz. For example, the high frequency signal can have a frequency of from about 1.2 kHz to about 100 kHz, or from about 1.5 kHz to about 100 kHz, or from about 2 kHz to about 50 kHz, or from about 3 kHz to about 20 kHz, or from about 3 kHz to about 15 kHz, or from about 5 kHz to about 15 kHz, or from about 3 kHz to about 10 kHz, or 1.5 kHz, 2 kHz, 3 kHz, 4 kHz, 5 kHz, 10 kHz, 15 kHz, 20 kHz, 50 kHz, or 100 kHz. In some embodiments, the high frequency electrical signal induces a motor response in the first muscle without inducing a motor response (or at least without inducing a patient-detectable motor response) in the second muscle.

The method 800 can continue at block 806 by administering a low frequency electrical signal to the target neural population to induce (e.g., selectively induce, preferentially induce, etc.) a motor response in the second muscle. The low frequency signal can have a frequency of less than 1.2 kHz. For example, the low frequency signal can have a frequency less than 1 kHz, less than 500 Hz, less than 200 Hz, less than 100 Hz, less than 50 Hz, less than 20 Hz, less than 10 Hz, less than 5 Hz, or less than 1 Hz. In some embodiments, the low frequency electrical signal induces a motor response in the second muscle without inducing a motor response (or at least without inducing a patient-detectable motor response) in the first muscle.

In some embodiments, the high frequency electrical signal and the low frequency electrical signal are administered at blocks 804 and 806, respectively, using the same electrode and/or electrodes. For example, the physician or programmer need not select different electrodes (or different electrode pairs) when switching between the high frequency signal and the low frequency signal. Rather, the high frequency electrical signal can preferentially activate the first muscle and the low frequency electrical signal can preferentially activate the second muscle by virtue of the frequency differential between the two signals, as described in Section 4.0. Likewise, in some embodiments the high frequency electrical signal and the low frequency electrical signal are administered at blocks 804 and 806 without repositioning (e.g., intra-operatively repositioning) the signal delivery device carrying the electrodes.

As provided above, the present technology also includes methods of programming a patient treatment system to perform some or all of the method 800. For example, the present technology includes a patient treatment system including a signal generator and a signal delivery element. The signal delivery element can be configured to be implanted proximate a target neural population including motor neurons associated with the first and second muscles. The signal generator can be programmed with instructions for generating the high frequency electrical signal and the low frequency electrical signal, and directing the high frequency electrical signal and the low frequency electrical signal to the signal delivery device.

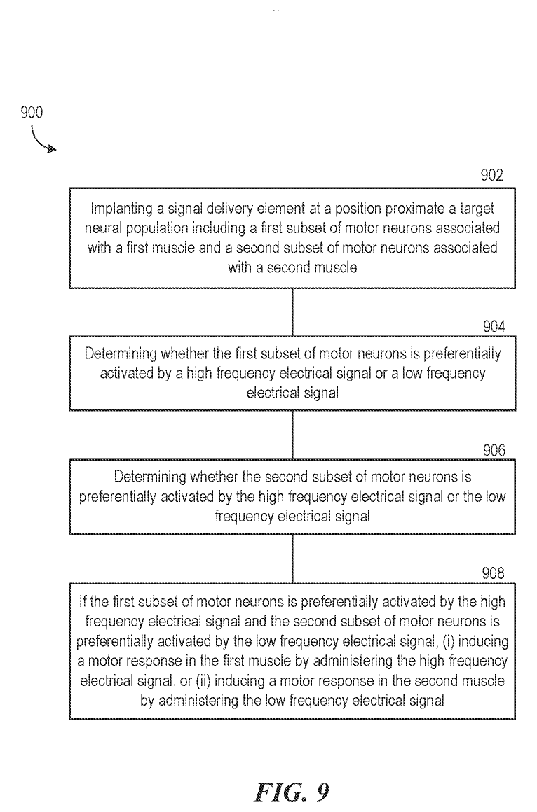

FIG. 9 is another block diagram illustrating a method 900 for treating a patient in accordance with embodiments of the present technology. Some or all of the blocks in the method 900 can be performed by a processor executing instructions stored on one or more elements of a patient treatment system. The method 900 can begin at block 902 by implanting a signal delivery element at a position proximate a target neural population, the target neural population including a first subset of motor neurons associated with a first muscle and a second subset of motor neurons associated with a second muscle. Block 902 can be generally similar to block 802 described with respect to FIG. 8.

The method 900 can continue at block 904 by determining whether the first subset of motor neurons is preferentially activated by a high frequency electrical signal and/or a low frequency electrical signal. This may include, for example, (i) administering the high frequency signal to the target neural population and detecting activity (e.g., EMG activity) at the first muscle, and (ii) administering the low frequency signal to the target neural population and detecting activity (e.g., EMG activity) at the first muscle. The method 900 can continue at block 906 by determining whether the second subset of neurons is preferentially activated by the high frequency electrical signal and/or the low frequency electrical signal. This may include, for example, (i) administering the high frequency signal to the target neural population and detecting activity (e.g., EMG activity) at the second muscle, and (ii) administering the low frequency signal to the target neural population and detecting activity (e.g., EMG activity) at the second muscle. In some embodiments, it is expected that the first subset of motor neurons (and thus the first muscle) will be preferentially activated by one of the high frequency electrical signal or the low frequency electrical signal, and the second subset of motor neurons (and thus the second muscle) will be preferentially activated by the other of the high frequency electrical signal or the low frequency electrical signal.

Based on the results of the determining blocks 904 and 906, the method 900 can continue by preferentially and/or selectively inducing a motor response in either the first muscle or the second muscle. For example, if the first subset of motor neurons is preferentially activated by the high frequency signal and the second subset of motor neurons is preferentially activated by the low frequency signal, then the method 900 can continue at block 908 by (i) inducing (e.g., preferentially inducing) a motor response in the first muscle by administering the high frequency electrical signal, or (ii) inducing (e.g., preferentially inducing) a motor response in the second muscle by administering the low frequency electrical signal. The high frequency signal and the low frequency signal can have any of the signal parameters described throughout this Detailed Description, such as under Section 7.0 below. In some embodiments, the high frequency electrical signal induces a motor response in the first muscle without inducing a motor response (or at least without inducing a patient-detectable motor response) in the second muscle, and/or the low frequency electrical signal induces a motor response in the second muscle without inducing a motor response (or at least without inducing a patient-detectable motor response) in the first muscle.

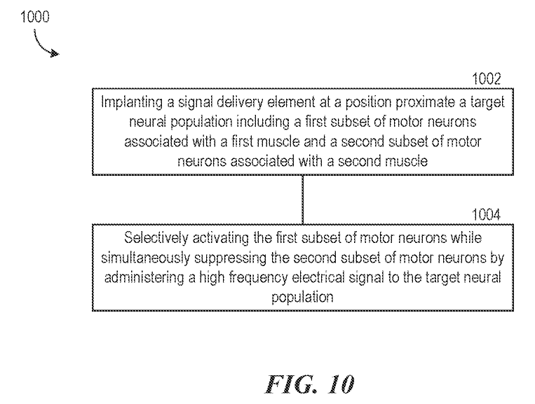

FIG. 10 is another block diagram illustrating a method 1000 for treating a patient in accordance with embodiments of the present technology. Some or all of the blocks in the method 1000 can be performed by a processor executing instructions stored on one or more elements of a patient treatment system. The method 1000 can begin at block 10002 by implanting a signal delivery element at a position proximate a target neural population, the target neural population including a first subset of motor neurons associated with a first muscle and a second subset of motor neurons associated with a second muscle. Block 1002 can be generally similar to block 802 described with respect to FIG. 8 and block 902 described with respect to FIG. 9.

The method 1000 can continue at block 1004 by selectively activating the first subset of motor neurons while simultaneously suppressing (e.g., inhibiting) the second subset of motor neurons by administering a high frequency electrical signal to the target neural population. For example, the high frequency signal may activate (e.g., excite) the first subset of motor neurons associated with the first muscle, leading to a motor response in the first muscle. At the same time, the high frequency signal may suppress (e.g., inhibit) the second subset of motor neurons associated with the second muscle, thereby minimizing or even preventing any motor response in the second muscle. In some embodiments, the high frequency signal may suppress the second subset of motor neurons by activating one or more inhibitory neurons or circuits associated with the second subset of motor neurons. The high frequency signal can have any of the signal parameters described throughout this Detailed Description, such as under Section 7.0 below.

As one skilled in the art will appreciate from the disclosure herein, FIGS. 8-10 are provided merely as examples of the types of methods that can be performed in accordance with the present technology. The present technology is not limited to the methods explicitly described herein. For example, certain operations included in the methods 800, 900, and 1000 can be omitted, and/or new operations can be added to the methods 800, 900, and 1000. Moreover, various operations in the methods 800, 900, and 1000 can be combined to form additional methods for treating patients.

### 6.0 Representative Clinical Applications

Without being bound by theory, embodiments of the present technology are expected to provide certain clinical benefits that may not be attainable using conventional therapeutic approaches. For example, the present technology can be utilized to treat motor disorders which may benefit from electrically inducing a motor response using electrical stimulation. For example, the present technology can be utilized to treat any motor or movement disorder, such as Parkinson's disease, spinal cord injury, stroke, essential tremor, dystonia, chorea/Huntington's disease, ataxia, tics and Tourette syndrome, restless leg syndrome, myoclonus, and the like. In such disorders, administering both a high frequency signal and a low frequency signal, as described with respect to the method 800 of FIG. 8, may recruit and activate more motor neurons and induce a motor response in more muscle groups. Of note, this can be achieved without changing which electrode(s) are delivering the signals, and without moving the signal delivery device (e.g., the lead carrying the electrode(s)). Without being bound by theory, this is expected to simplify treating various motor dysfunctions that benefit from activation of multiple muscles. For example, rather than having to reprogram which electrodes are delivering the signal and/or move the signal delivery device, a user (e.g., a physician, programmer, or patient) can simply change the frequency of the signal being administered.

Furthermore, embodiments of the present technology may be useful in treating certain conditions associated with spontaneous firing neurons. For example, as described herein, high frequency signals can have an inhibitory or suppressive effect on certain motor neuron populations. Accordingly, high frequency signals can be administered to a patient to suppress (e.g., reduce) spontaneous firing of motor neurons in conditions such as epilepsy, dystonia, essential tremor, Parkinson's disease, spasticity from spinal cord injury, depression, autism, or the like. The high frequency signals may provide additional therapeutic benefit to such patients beyond quieting spontaneous active neurons. For example, the high frequency signals may also beneficially activate certain motor neurons (e.g., those that aren't spontaneously active), provide pain relief, or induce other therapeutically advantageous effects. Of course, other clinical applications of the present technology may exist beyond those expressly recited herein. Therefore, the present technology is not limited to treating the indications recited herein.

In some embodiments, the present technology can be applied to other neural structures beyond the spinal cord. For example, the present technology can be utilized for modulating neurons located in the patient's brain (e.g., deep brain structures) or the patient's peripheral nervous system. Likewise, although primarily described in the context of preferentially activating or inhibiting motor neurons and/or motor responses, the present technology may also be utilized to preferentially activate or inhibit other neural fibers, such as any neural fibers involved in inhibitory circuits. Accordingly, the present technology may be utilized to address other conditions beyond those expressly disclosed herein.

### 7.0 Representative Signal Delivery Parameters

The electrical signals described above may be delivered in accordance with several suitable signal delivery parameters. For example, the high frequency signals described herein may have a frequency between from about 1.2 kHz to about 500 kHz, or from about 1.2 kHz to about 100 kHz, or from about 1.5 kHz to about 100 kHz, or from about 2 kHz to about 50 kHz, or from about 3 kHz to about 20 kHz, or from about 3 kHz to about 15 kHz, or from about 5 kHz to about 15 kHz, or from about 3 kHz to about 10 kHz, or 1.5 kHz, 2 kHz, 3 kHz, 4 kHz, 5 kHz, 10 kHz, 15 kHz, 20 kHz, 50 kHz, or 100 kHz. In particular embodiments, representative current amplitudes for the high frequency therapy signals are from 0.1 mA to 20 mA, or 0.5 mA to 10 mA, or 0.5 mA to 7 mA, or 0.5 mA to 5 mA. Representative pulse widths for the high frequency signals range from about 10 to about 333 microseconds, about 10 to about 166 microseconds, about 25 to about 166 microseconds, about 20 to about 100 microseconds, about 30 to about 100 microseconds, about 30 to about 40 microseconds, about 10 to about 50 microseconds, about 20 to about 40 microseconds, about 25 to about 35 microseconds, about 30 to about 35 microseconds, and about 30 microseconds. Duty cycles can range from about 10% to about 100%, and in a particular duty cycle, signals are delivered for 20 seconds and interrupted for 2 minutes (an approximate 14% duty cycle). In other embodiments, these parameters can have other suitable values. Other suitable parameters and other therapy features are disclosed in the following materials, each of which is incorporated by reference: U.S. Patent Application Publication No. US2009/0204173; U.S. Patent Application Publication No. US2014/0296936; and U.S. Patent Application Publication No. US2010/0274314.

The low frequency signals described herein may have a frequency less than 1.2 kHz. For example, the low frequency signal can have a frequency less than 1 kHz, less than 500 Hz, less than 200 Hz, less than 100 Hz, less than 50 Hz, less than 20 Hz, less than 10 Hz, less than 5 Hz, or less than 1 Hz, such as 0.1 Hz, 0.2 Hz, 0.3 Hz, 0.4 Hz, 0.5 Hz, or the like. Representative pulse widths range from about 50 to about 666 microseconds, about 80 microseconds to about 333 microseconds, about 80 to about 166 microseconds, about 80 to about 120 microseconds, or about 100 microseconds. In particular embodiments, representative current amplitudes for the therapy signal are from 0.1 mA to 20 mA, or 0.5 mA to 10 mA, or 0.5 mA to 7 mA, or 0.5 mA to 5 mA. In other embodiments, these parameters can have other suitable values.

In some embodiments, the high frequency signals, the low frequency signals, or both the high frequency signals and the low frequency signals do not produce paresthesia when delivered to the patient, and can therefore be referred to as “non-paresthesia producing electrical signals” or “paresthesia-free electrical signals.” Paresthesia-free signals may have combinations of frequency, pulse widths, amplitudes, and/or duty cycles that cause the signal to be below a patient’s sensory perception threshold. For example, paresthesia-free electrical signals may have a frequency of between about 1 Hz and 100 kHz, or between about 1.2 kHz and about 100 kHz. Additional examples of paresthesia-free electrical signals are described in U.S. Patent Application Publication No. US2010/0274314, previously incorporated by reference herein. In other embodiments, the high frequency signals, the low frequency signals, or both the high frequency signals and the low frequency signals described herein may induce paresthesia when delivered to the patient.

### 8.0 Representative Examples

The following examples are provided to further illustrate embodiments of the present technology and are not to be interpreted as limiting the scope of the present technology. To the extent that certain embodiments or features thereof are mentioned, it is merely for purposes of illustration and, unless otherwise specified, is not intended to limit the present technology. It will be understood that many variations can be made in the procedures described herein while still remaining within the bounds of the present technology. Such variations are intended to be included within the scope of the presently disclosed technology.

1. A method for inducing a motor response in a patient, comprising:

implanting a signal delivery device at a position proximate a target neural population in the patient, the target neural population including motor neurons associated with a first muscle and a second muscle;

administering a first electrical signal to the target neural population via the signal delivery device to induce a motor response in the first muscle, the first electrical signal having a frequency in a frequency range of from 1.2 kHz to 100 kHz; and

administering a second electrical signal to the target neural population via the signal delivery device to induce a motor response in the second muscle, the second electrical signal having a frequency of less than 1.2 kHz.

2. The method of example 1 wherein administering the first electrical signal preferentially induces a motor response in the first muscle relative to any motor response induced in the second muscle.

3. The method of example 1 or 2 wherein administering the first electrical signal induces a motor response in the first muscle without inducing a patient-detectable motor response in the second muscle.

4. The method of any of examples 1-3 wherein administering the second electrical signal preferentially induces a motor response in the second muscle relative to any motor response induced in the first muscle.

5. The method of any of examples 1-4 wherein administering the second electrical signal induces a motor response in the second muscle without inducing a patient-detectable motor response in the first muscle.

6. The method of any of examples 1-5 wherein administering both the first electrical signal and the second electrical signal increases the overall motor response in the patient relative to administering only the first electrical signal or only the second electrical signal.

7. The method of any of examples 1-6 wherein the signal delivery device includes at least one electrode, and wherein administering the first electrical signal and the second electrical signal includes administering the first electrical signal and the second electrical signal from the same at least one electrode.

8. The method of example 7 wherein the at least one electrode includes a pair of electrodes, and wherein the first electrical signal and the second electrical signal are administered via the same pair of electrodes.

9. The method of any of examples 1-8 wherein the first electrical signal and the second electrical signal are administered without repositioning the signal delivery device.

10. The method of any of examples 1-9 wherein the first electrical signal has a frequency in a frequency range of from about 5 kHz to about 15 kHz.

11. The method of any of examples 1-10 wherein the first electrical signal has a frequency of about 10 kHz.

12. The method of any of examples 1-11 wherein the second electrical signal has a frequency of less than 100 Hz.

13. The method of any of examples 1-12 wherein the second electrical signal has a frequency of less than 10 Hz.

14. The method of any of examples 1-13 wherein the second electrical signal has a frequency of less than 1 Hz.

15. The method of any of examples 1-14 wherein the patient is diagnosed with an indication characterized by motor dysfunction.

16. A method for treating a patient, comprising:

implanting a signal delivery device at a position proximate a target neural population, the target neural population including a first subset of motor neurons associated with a first muscle and a second subset of motor neurons associated with a second muscle;

determining whether the first subset of motor neurons is preferentially activated by (i) a first electrical signal having a frequency in a frequency range of from 1.2 kHz to 100 kHz, or (ii) a second electrical signal having a frequency less than 1.2 kHz; and

determining whether the second subset of motor neurons is preferentially activated by the first electrical signal or the second electrical signal,

wherein the first subset of motor neurons is preferentially activated by one of the first electrical signal or the second electrical signal, and wherein the second subset of motor neurons is preferentially activated by the other of the first electrical signal or the second electrical signal.

17. The method of example 16, wherein, if the first subset of motor neurons is preferentially activated by the first electrical signal and the second subset of motor neurons is preferentially activated by the second electrical signal, the method further comprises:

inducing a motor response in the first muscle by administering the first electrical signal to the target neural population via the signal delivery device; or

inducing a motor response in the second muscle by administering the second electrical signal to the target neural population via the signal delivery device.

18. The method of example 16 or 17 wherein determining whether the first subset of motor neurons is preferentially activated by the first electrical signal or the second electrical signal includes:

administering the first electrical signal to the target neural population via the signal delivery device;

detecting a motor response to the first electrical signal at the first muscle;

administering the second electrical signal to the target neural population via the signal delivery device;

detecting a motor response to the second electrical signal at the first muscle; and

comparing the motor response to the first electrical signal and the motor response to the second electrical signal.

19. The method of example 18 wherein the first electrical signal and the second electrical signal are administered from the same one or more electrodes.

20. The method of any of examples 14-16 wherein determining whether the second subset of motor neurons is preferentially activated by the first electrical signal or the second electrical signal includes:

administering the first electrical signal to the target neural population via the signal delivery device;

detecting a motor response to the first electrical signal at the second muscle;

administering the second electrical signal to the target neural population via the signal delivery device;

detecting a motor response to the second electrical signal at the second muscle; and

comparing the motor response to the first electrical signal and the motor response to the second electrical signal.

21. The method of example 20 wherein the first electrical signal and the second electrical signal are administered from the same one or more electrodes.

22. The method of any of examples 16-21 wherein the first electrical signal has a frequency in a frequency range of from about 5 kHz to about 15 kHz.

23. The method of any of examples 16-22 wherein the first electrical signal has a frequency of about 10 kHz.

24. The method of any of examples 16-23 wherein the second electrical signal has a frequency of less than 100 Hz.

25. The method of any of examples 16-24 wherein the second electrical signal has a frequency of less than 10 Hz.

26. The method of any of examples 16-25 wherein the second electrical signal has a frequency of less than 1 Hz.

27. The method of any of examples 16-26 wherein the patient is diagnosed with an indication characterized by motor dysfunction.

28. A method for treating a patient, comprising:

implanting a signal delivery device at a position proximate a target neural population in the patient, the target neural population including a first subset of motor neurons associated with a first muscle and a second subset of motor neurons associated with a second muscle; and

selectively activating the first subset of motor neurons while simultaneously suppressing the second subset of motor neurons by administering an electrical signal to the target neural population via the signal delivery device, the electrical signal having a frequency in a frequency range of from 1.2 kHz to 100 kHz.

29. The method of example 28 wherein activating the first subset of motor neurons induces a patient-detectable motor response in the first muscle, and wherein suppressing the second subset of motor neurons inhibits any patient-detectable motor response in the second muscle.

30. The method of example 28 or 29 wherein the electrical signal has a frequency in a frequency range of from about 5 kHz to about 15 kHz.

31. The method of any of examples 28-30 wherein the electrical signal has a frequency of about 10 kHz.

32. The method of any of examples 28-31 wherein the second subset of motor neurons include spontaneously firing neurons, and wherein the electrical signal at least partially suppresses the spontaneously firing neurons.

33. The method of any of examples 28-32 wherein the patient is diagnosed with an indication characterized by motor dysfunction.

34. A method for treating a patient, comprising:

programming a signal generator to:

administer a first electrical signal to a target neural population via an implanted signal delivery device, the target neural population including motor neurons associated with a first muscle and a second muscle, wherein the first electrical signal has a frequency in a frequency range of from 1.2 kHz to 100 kHz, and wherein the first electrical signal induces a motor response in the first muscle; and

administer a second electrical signal to the target neural population via the implanted signal delivery device, wherein the second electrical signal has a frequency of less than 1.2 kHz, and wherein the second electrical signal induces a motor response in the second muscle.

35. A patient treatment system, comprising:

a signal delivery device configured to be implanted at a position proximate a target neural population in the patient, the target neural population including motor neurons associated with a first muscle and a second muscle; and

a signal generator having a computer readable storage medium with instructions that, when executed, cause the signal generator to:

deliver a first electrical signal to the target neural population via the signal delivery device to induce a motor response in the first muscle, the first electrical signal having a frequency in a frequency range of from 1.2 kHz to 100 kHz; and

deliver a second electrical signal to the target neural population via the signal delivery device to induce a motor response in the second muscle, the second electrical signal having a frequency less than 1.2 kHz.

### 7.0 Conclusion

From the foregoing, it will be appreciated that specific embodiments of the disclosed technology have been described herein for purposes of illustration, but that various modifications may be made without deviating from the technology. For example, therapy signals described herein can be delivered at combinations of parameter values within the foregoing ranges at values that are not expressly disclosed herein. Certain aspects of the technology described in the context of particular embodiments may be combined or eliminated in other embodiments. For example, the therapy signal can be monophasic with a passive charge elimination phase. In some embodiments, the foregoing techniques can be used to address patient deficits than pain. Further, while advantages associated with certain embodiments of the disclosed technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

The use of “and/or” in reference to a list of two or more items is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Where a range of values is provided, it is understood that each intervening value, to the tenth of the unit of the lower limit unless the context clearly dictates otherwise, to between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

1. A method for treating a patient, comprising:
implanting a signal delivery device at a position proximate a target neural population in the patient, the target neural population comprises a first subset of motor neurons associated with a first muscle and a second subset of motor neurons associated with a second muscle; and
selectively activating the first subset of motor neurons while simultaneously suppressing the second subset of motor neurons by administering an electrical signal to the target neural population via the signal delivery device.

2. The method of claim 1, wherein the electrical signal has a frequency in a frequency range of from 1.2 kHz to 100 kHz.

3. The method of claim 1, wherein selectively activating the first subset of motor neurons comprises inducing a patient-detectable motor response in the first muscle.

4. The method of claim 1, wherein suppressing the second subset of motor neurons comprises inhibiting any patient-detectable motor response in the second muscle.

5. The method of claim 1, wherein the electrical signal has a frequency in a frequency range of from about 5 kHz to about 15 kHz.

6. The method of claim 1, wherein the electrical signal has a frequency of 10 kHz.

7. The method of claim 1, wherein the second subset of motor neurons comprises spontaneously firing neurons.

8. The method of claim 7, wherein the electrical signal at least partially suppresses the spontaneously firing neurons.

9. The method of claim 1, wherein the patient has an indication characterized by motor dysfunction.

10. The method of claim 1, wherein the electrical signal is a non-paresthesia producing electrical signal.
